(19) United States
(12) Patent Application Publication
(10) Pub. No.: US 2026/0337674 A1
(43) Pub. Date: Oct. 29, 2026

(54) METHOD FOR IN VITRO OR EX VIVO DETECTION OF AN IMMUNOCOMPROMISED STATUS IN A SUBJECT

(71) Applicants: bioMérieux, MARCY L'ETOILE (FR); HOSPICES CIVILS DE LYON, LYON (FR); UNIVERSITE CLAUDE BERNARD LYON 1, VILLEURBANNE (FR)

(72) Inventors: Sophie BLEIN, CIVRIEUX D'AZERGUES (FR); Karen BRENGEL-PESCE, LES ABREST EN DAUPHINE (FR); Elisabeth CERRATO, LYON (FR); Guillaume MONNERET, LYON (FR); Estelle PERONNET, LYON (FR); Fabienne VENET, LYON (FR)

(73) Assignees: bioMérieux, MARCY L'ETOILE (FR); HOSPICES CIVILS DE LYON, LYON (FR); UNIVERSITE CLAUDE BERNARD LYON 1, VILLEURBANNE (FR)

(21) Appl. No.: 19/110,307

(22) PCT Filed: Sep. 8, 2023
(86) PCT No.: PCT/FR2023/051359

§ 371 Date: Mar. 10, 2025

(30) Foreign Application Priority Data
Sep. 9, 2022 (FR) FR22/09043

(51) Int. Cl. C12Q 1/686; C12Q 1/48; C12Q 1/6809; C12Q 1/6813; C12Q 1/6869; C12Q 1/6876

(52) U.S. Cl. CPC C12Q 1/686; C12Q 1/48; C12Q 1/6809; C12Q 1/6813; C12Q 1/6869; C12Q 1/6876; C12Y 207/07; C12Q 2600/158; C12Q 2600/16; C12Q 2600/166; G01N 2800/24; G01N 2800/60

(57) ABSTRACT

A method for the in vitro or ex vivo detection of the presence or absence of an immunocompromised status in a subject includes a step of quantifying, in a biological sample from said subject, the expression of at least 2 target genes selected from the group made of TAP2, C3AR1, CD177 and IL1R2, and a step of comparing the expression of each of the genes respectively to a reference value so as to identify whether or not a variation in expression is present.

### PRIOR ART

Sepsis affects nearly 50 million people worldwide every year, more than 40% of whom are children under the age of 5. It is one of the leading causes of death worldwide, with more than 11 million deaths each year. However, the majority of these deaths are preventable. In Europe, it is estimated that sepsis is responsible for nearly 700 000 deaths each year, including nearly 57 000 deaths in France with an average cost of around €16 000 per hospitalization.

Sepsis is a systemic inflammatory response to an infection (bacterial, viral, fungal or parasitic) that is defined as an acute state of dysregulation of the body's response to this infection, leading to the loss of organ function and a life-threatening risk to the patient, the most severe form of which is septic shock. Sepsis is accompanied by an exacerbated production of inflammatory mediators. For example, reference is made to a “cytokine storm” to evoke the massive production of cytokines (chemical mediators that allow communication between our cells), which affect the functioning of vital organs in an acute way, and can lead to functional after effects in the longer term. Patients with sepsis are usually admitted to intensive care, where they in particular receive antibiotics and the supports needed to maintain vital functions. However, for more than twenty years, despite considerable progress made in understanding the pathophysiology associated with sepsis, no new sepsis-specific therapy has emerged.

The high heterogeneity of patients is pointed out as the main reason for the failure of decades of treatment strategies, including anti-inflammatory strategies, in sepsis. This emphasizes the crucial need for patient stratification and for a more individualized approach.

As regards the pathophysiology of sepsis, after the initial inflammation, a persistent immunosuppression has been described several times and has been found to have adverse consequences on patients (increase in the risk of nosocomial infection, in the risk of mortality and in the length of stay) and on health care costs.

Consequently, adjuvant immunostimulation is now envisioned to counterbalance this immunosuppressive response and to restore immune functions in patients. However, this new approach is based on the ability to identify or enrich patient groups with the most immunosuppressed functions. Since immunosuppression has no clinical sign, patient stratification must be based on immune biomarkers.

Monocytes are by nature extremely “plastic” cells. Thus, throughout the progression of a disease, the monocytes can alternately be pro- and anti-inflammatory cells. The result of these pro- and anti-inflammatory forces that act on monocytes at a given time can be broadly reflected by the level of expression of mHLA-DR at their surface.

Consequently, one of the reference tests for monitoring immune changes in patients in intensive care units (e.g. patients with sepsis, with trauma, having undergone major surgery, with burns, or patients with pancreatitis) is the decrease in expression of a surface receptor belonging to the major histocompatibility complex (MHC) class II, namely HLA-DR (human leukocyte antigen-D related), on the surface of the monocytes (mHLA-DR), measured by flow cytometry. Indeed, this marker provides valuable information in terms of predicting mortality or assessing the risk of secondary infections in these patients (Venet et al. (2018) Nat Rev Nephrol 2018; 14:121-137).

Measurement of mHLA-DR expression is a known method in the literature for identifying whether or not a patient, for example with sepsis, is immunocompromised (Monneret and Venet (2016) Cytometry Part B (Clinical Cytometry) 90B: 376-386). By virtue of a standardized measurement, a clinical decision threshold has been proposed, namely an mHLA-DR of at least 5000 antibodies bound per cell (Ab/C), such as for example 8000 Ab/C, and used to identify immunocompromised patients in intensive care.

To date, the measurement of mHLA-DR is based on flow cytometry, a technology that cannot be implemented in all hospital sites for clinical routine due to many limitations. Indeed, flow cytometry presents pre-analytical constraints and requires qualified technicians to exploit and interpret the results. Moreover, the deficiencies in terms of the installation of flow cytometers in hospitals, when they are present, is not compensated for by 24 h/24 and 7d/7 accessibility (Monneret and Venet (2014), Crit Care 18:102). Finally, it is a technology that requires time, which is rarely available when the patient is in a resuscitation unit or intensive care unit.

To overcome these drawbacks, other biomarkers, using molecular biology tools, have been proposed. For example, mention may be made of a biomarker based on the ratio of the level of expression, at the messenger RNA (mRNA) level, of CD74 on day D3 (following admission of the patient to a medical facility) to the level of expression of CD74 on D1. CD74 represents the invariant γ-chain of HLA-DR. It has been shown that the CD74 D3/D1 expression ratio is associated with the appearance of secondary infections acquired in intensive care (Peronnet et al. (2017) Intensive Care Medicine; 43 (7): 1013-20).

Document WO2013/156627 also proposes a method for determining the immunocompromised or non-immunocompromised status, from the determination of the anellovirus load, in a biological sample.

In parallel, a prototype multiplex molecular tool making it possible to evaluate, on an automated platform, the expression of a set of 16 biomarkers, in particular mRNAs, was implemented to identify the immune profile of patients. (Tawfik et al. (2020) JID 2020:222 (Suppl 2)). However, the biomarkers are not derived from a dedicated cohort study and are therefore not reliable enough to distinguish and stratify patients. In addition, the performances obtained need to be improved and no correlation is made between the expressions of the various biomarkers and a value of mHLA-DR characteristic of immunocompetence.

To date, several genes are identified as a potential link with immune status in a subject. By way of example,
the TAP2 (Antigen Peptide Transporter 2) gene codes for a membrane-associated protein of the ABC transporter superfamily (ATP-binding cassette), which is involved in the transport of peptides from the cytoplasm to the endoplasmic reticulum as part of the antigen presentation process of class I molecules. Expression of the transcript of this gene has, in addition, been studied in septic patients, in whom it has been shown to enable the identification of patients with a particular endotype associated with the occurrence of death at 28 days (Scicluna et al. (2017), Lancet Respir Med 5 (10): 816-826);
the C3AR1 (complement component 3a receptor 1) gene codes for the receptor for complement C3a, a protein released during complement activation. The binding of the anaphylatoxin C3a to its receptor activates chemotaxis and opsonization of bacteria. In the literature, it has been shown that the C3AR1 gene transcript was more greatly expressed in septic patients than in healthy volunteers (Napier et al. (2016) J. Exp. Med. 213 (11): 2365-2382);
the CD177 gene codes for the neutrophil membrane glucoprotein CD177, discovered in 1971 in a case of neonatal neutropenia. An increase in the expression level of CD177, at the mRNA and protein level, in circulating neutrophils of patients who had experienced septic shock was demonstrated in a study (Demaret et al. (2016), Immunol. Letters 178:122-130);
the IL1R2 gene codes for a receptor for interleukin-1, and more specifically interleukin-1a (IL1A) and interleukin-1β (IL1B), which transmit intracellular signals via pathways shared by other receptors, such as the interleukin-18 (IL18) receptor, leading to the secretion of inflammation-mediating factors. The IL1R2 receiver is described as a “decoy” receptor, which binds IL1A and IL1B and therefore prevents them from binding to their regular receptors, thereby resulting in inhibition of the signal transduction normally mediated by these interleukins. The IL1R2 transcript has been identified as being differentially expressed in the neutrophils of septic patients in a bioinformatics study (He et al. (2019) 35 (6): 481-490). IL1R2 has been proposed as a diagnostic biomarker for sepsis, on the basis of results of measuring the serum concentration of IL1R2 in healthy volunteers and septic patients (Lang et al. (2017) Shock 47 (1): 119-124). Chromatin immunoprecipitation analyses have shown differences at the chromatin level (histone acetylation and methylation) of the IL1R2 gene promoter between septic patients and healthy volunteers (Weiterer et al. (2015) PLOS One 10 (3): e0121748).

Although these markers are described individually, no study has to date demonstrated a sufficiently robust link between their combined expression and the immunocompromised nature of patients, nor has any correlation been demonstrated between these markers and commonly used immunosuppression markers, such as HLA-DR expression at the surface of monocytes, percentage of regulatory T cells or the number of CD4+ T cells (Demaret et al. (2016) Immunology Letters 178:122-130). In other words, there is no alternative to flow cytometry techniques.

There is therefore a clear need to identify a reliable (i.e. with acceptable sensitivity and/or specificity) and fast means, which is also easy to read/interpret, and simple to use and can be integrated in clinical routine, in order to decide on the immune status of the patient, in particular to identify immunosuppression.

### SUMMARY OF THE INVENTION

A first object concerns a method for the in vitro or ex vivo detection of an immunocompromised status in a subject, comprising the following steps:
a. quantifying, in a biological sample from said subject, the expression of at least 2 target genes selected from the group consisting of TAP2, C3AR1, CD177 and IL1R2;
b. comparing the expression of each of the genes respectively with a reference value so as to identify the presence or absence of a variation in the expression, the reference value corresponding to the expression of said target gene in a subject having a normal immune status defined by an mHLA-DR greater than 5000 cell-bound antibodies (Ab/C), preferably greater than 8000 cell-bound antibodies (Ab/C), the immunocompromised status being detected on the basis of at least two variations in the expression, said variations being selected from:
   an underexpression of TAP2,
   an overexpression of C3AR1,
   an overexpression of CD177, and
   an overexpression of IL1R2.

According to a particular embodiment, step a) comprises quantifying the expression of at least 3 target genes selected from the group consisting of TAP2, C3AR1, CD177 and IL1R2.

According to another particular embodiment, step a) comprises quantifying the expression of the TAP2 target gene and at least one other target gene selected from C3AR1, CD177 and IL1R2.

According to another particular embodiment, step a) comprises quantifying the expression of the 4 target genes TAP2, C3AR1, CD177 and IL1R2.

Advantageously, the method according to the invention further comprises a step of quantifying the expression of at least one other target gene selected from the group consisting of CD3D, CIITA, CD74, CTLA4, CX3CR1, IFNγ and S100A9, wherein the immunocompromised status is detected taking into account at least one additional variation in the expression, said additional variation being selected from:
an underexpression of CD3D,
an underexpression of CIITA,
an underexpression of CD74,
an underexpression of CTLA4,
an underexpression of CX3CR1,
an overexpression of IFNγ, and
an overexpression of S100A9.

According to a particular embodiment, the biological sample is a blood sample, preferably a whole blood sample.

According to another particular embodiment, the biological sample is from a patient in the emergency department, the resuscitation unit, in an intensive care unit (ICU) or in an ongoing care unit, preferably from an ICU patient.

According to another particular embodiment, the expression is measured at the RNA or messenger RNA level, preferably by RT-PCR, preferably RT-qPCR, by sequencing or by hybridization.

According to a particular embodiment, the expression is normalized with respect to the expression of one or more housekeeping genes selected from the group consisting of DECR1, HPRT1, PPIB, GAPDH and ACTB.

Another subject of the invention relates to a kit for use thereof in detecting an immunocompromised status in a subject, said kit comprising means for amplifying and/or means for detecting the expression of at least 2 target genes, preferably at least 3 target genes, selected from the group consisting of TAP2, C3AR1, CD177 and IL1R2. Advantageously, the kit comprises means for amplifying and/or means for detecting the expression of 4 target genes TAP2, C3AR1, CD177 and IL1R2.

According to a particular embodiment, the kit further comprises means for amplifying and/or means for detecting the expression of at least one other target gene selected from the group consisting of CD3D, CIITA, CD74, CTLA4, CX3CR1, IFNγ and S100A9.

Advantageously, the amplification and/or detection means are reagents specific for the expression products of said target genes, selected from amplification primers or hybridization probes.

### DETAILED DESCRIPTION OF THE INVENTION

After much research, it is to the credit of the inventors to have been able to identify, unexpectedly and surprisingly, a signature of several genes of which the dysregulated expression is characteristic of an immunocompromised status.

An objective of the present invention is to identify the same immunocompromised patients, particularly in intensive care units, as those characterized by an mHLA-DR expression of <8000 bound antibodies/cell (Ab/C).

Thus, a first subject of the present invention relates to a method for the in vitro or ex vivo detection of an immunocompromised status in a subject, comprising the following steps:
a. quantifying, in a biological sample from said subject, the expression of at least 2 target genes selected from the group consisting of TAP2, C3AR1, CD177 and IL1R2;
b. comparing the expression of each of the genes respectively with a reference value so as to identify the presence or absence of a variation in the expression.

The present invention therefore has various advantages, in particular the fact that it allows a generalization of the immunosurveillance of subjects, such as that of patients in intensive care, simply and rapidly by means of a technology (tools and method) that can be implemented at all hospital sites, unlike flow cytometry. The present invention also makes it possible to identify immunocompromised patients, for whom it would be relevant to administer immunostimulant therapies in the context of clinical trials, thus to demonstrate the efficacy of such therapies. In addition, the invention opens the door to personalized medicine in patients, especially those in intensive care.

To the knowledge of the inventors, it has never been described or suggested that the demonstration of a variation in the expression of at least 2 target genes selected from the group consisting of the TAP2, C3AR1, CD177 and IL1R2 genes makes it possible to detect, in vitro or ex vivo, an immunocompromised status in a subject. Moreover, it has never been described or suggested that the result of this detection correlates with a quantification of HLA-DR expression at the surface of monocytes (mHLA-DR) of less than 8000 Ab/C, advantageously less than 7500 Ab/C, less than 7500 Ab/C, preferably less than 7000 Ab/C, less than 6500 Ab/C, or even less than 6000 Ab/C, less than 5500 Ab/C or even less than 5000 Ab/C, measured by flow cytometry.

The target genes of which the expression is quantified in the method which is the subject of the present invention are known to a person skilled in the art and the chromosomal locations are given in particular in table 1 below.

TABLE 1

| Biomarkers | Chromosomal location (GRCh38/hg38) |
|---|---|
| TAP2 | chr6: 32,821,833-32,838,823 |
| C3AR1 | chr12: 8,058,302-8,066,471 |
| CD177 | chr19: 43,353,659-43,363,172 |
| IL1R2 | chr2: 101,991,816-102,028,544 |

In the context of the invention, and in contrast to a normal immune status, described as immunocompetent, the expressions “immunocompromised”, “immunosuppressed”, “immunodeficient”, “hypoactive immune status” or “immune paralysis” are used interchangeably and denote a less active immune system/immune response than normal in response to an infection or inflammatory condition. In particular, it is an immunocompromised state characterized by impaired innate and adaptive immune responses, in particular increased apoptosis and lymphocyte dysfunction, impaired phagocytic functions, monocyte deactivation with a decrease in surface expression of HLA class II and impaired cytokine production.

In the context of the invention, the term “subject” denotes a human being, and the subject is preferably a patient. The patient is defined as a person who has been in contact with a healthcare professional, particularly a doctor, or a medical structure or healthcare facility.

According to a particular embodiment, the subject is a patient in a healthcare facility, preferably in a hospital, more preferably in the emergency department, resuscitation department, intensive care unit (ICU) or ongoing care unit; most particularly, the subject is a patient in the ICU.

In the context of the invention, the terms “biomarker” and “marker” are used interchangeably and denote an objectively measurable biological characteristic that represents an indicator of normal or pathological biological processes. In particular, it may be a molecular biomarker, preferably detectable at mRNA level. More particularly, the biomarker may be an endogenous biomarker or loci, such as an HERV or a gene.

“Sepsis” is a pathological condition in which the immune response is dysregulated in an individual, following infection, leading to multiple and potentially fatal organ dysfunctions and failure. “Septic shock” is a subtype of sepsis, in which hypotension persists, despite appropriate vascular filling.

In the context of the invention, the expression “biological sample” designates any sample originating from a subject, and which may be of different natures, such as blood or blood derivatives, sputum, urine, stools, skin, cerebrospinal fluid, bronchoalveolar lavage fluid, abdominal cavity puncture fluid, saliva, gastric secretions, semen, seminal fluid, tears, spinal cord, trigeminal nerve ganglion, adipose tissue, lymphoid tissue, placental tissue, gastrointestinal tract tissue, genital tract tissue, central nervous system tissue. In particular, this sample may be a biological fluid, such as a blood sample or a sample derived from blood, which may in particular be selected from whole blood (such as collected from the venous line, i.e. containing white and red cells, platelets and plasma), plasma, serum, and also any type(s) of cells extracted from blood, such as peripheral blood mononuclear cells (or PBMCs, containing lymphocytes (B, T and NK cells), dendritic cells and monocytes), subpopulations of B cells, purified monocytes, or neutrophils.

In the context of the invention, the expressions “reference value”, “normal value”, “norm”, “usual value” or “reference biological sample” are used interchangeably and denote a value or an average of the values of a parameter (e.g. gene expression, in particular at the mRNA level, . . . ) derived from one or more healthy subjects, with a normal or immunocompetent immune status, i.e. a subject in whom the immune status is not immunocompromised, in particular with an mHLA-DR value greater than 5000 Ab/C, in particular greater than 5500 Ab/C, greater than 6000 Ab/C, greater than 6500 Ab/C, or even greater than 7000 Ab/C, greater than 7500 Ab/C, and most particularly greater than 8000 Ab/C, and who does not have an immune response impaired as a result of inflammation or sepsis, as opposed to a “tested value”, “test value” or “biological test sample”. The reference value and the test value are obtained by implementing the same method of detection, quantification, identification, etc.

In the context of the invention, the expression “variation in expression” designates an overexpression or underexpression of the gene. In particular:
the term “overexpression” means that the expression of a gene is increased relative to a reference value, that is to say obtained in an immunocompetent subject defined by an mHLA-DR value greater than 5000 Ab/C, in particular greater than 5500 Ab/C, advantageously greater than 6000 Ab/C, in particular greater than 6500 Ab/C, preferably greater than 7000 Ab/C, in particular greater than 7500 Ab/C, and most particularly by an mHLA-DR value greater than 8000 Ab/C;
the term “underexpression” means that the expression of a gene is decreased relative to a reference value, that is to say obtained in an immunocompetent subject defined by an mHLA-DR value greater than 5000 Ab/C, in particular greater than 5500 Ab/C, advantageously greater than 6000 Ab/C, in particular greater than 6500 Ab/C, preferably greater than 7000 Ab/C, in particular greater than 7500 Ab/C, and most particularly by an mHLA-DR value greater than 8000 Ab/C.

The terms “coding” or “coding for”, “codes” or “codes for” are used interchangeably and refer to the property inherent in the specific nucleotide sequences in a polynucleotide, such as a gene, a cDNA or an mRNA, to serve as a template for the synthesis of other polymers having a defined amino acid sequence, and the biological properties resulting therefrom. Thus, a gene codes for a protein if the transcription and translation of the mRNA corresponding to this gene produce the protein in a cell or other biological system. Both the coding strand, the nucleotide sequence of which is identical to the mRNA sequence and which is generally described in sequence listings and databases, and the non-coding strand, used as a template for transcription of a gene or of the cDNA, may be denoted as coding for the protein or other product of this gene or cDNA.

In the context of the invention, the expression “amplification primer” denotes a nucleotide fragment which may comprise from 5 to 100 nucleotides, preferably from 15 to 30 nucleotides, and which possesses a specificity of hybridization with a target nucleotide sequence, under conditions determined for the initiation of an enzymatic polymerization, for example in a reaction for enzymatic amplification of the target nucleotide sequence. Generally use is made of “pairs of primer” consisting of two primers. When it is desired to amplify several different genes, several different pairs of primers are preferably used, each preferentially having the ability to hybridize specifically with a different gene.

In the context of the invention, the expression “hybridization probe” denotes a nucleotide fragment typically comprising from 5 to 100 nucleotides, preferably from 15 to 90 nucleotides, even more preferably from 15 to 35 nucleotides, having hybridization specificity under conditions determined for forming a hybridization complex with a target nucleotide sequence. The probe also comprises a reporter (such as a fluorophore, an enzyme or any other detection system) which will enable the detection of the target nucleotide sequence. In the present invention, the target nucleotide sequence may be a nucleotide sequence comprised in a messenger RNA (mRNA) or a nucleotide sequence comprised in a complementary DNA (cDNA) obtained by reverse transcription of said mRNA. When it is desired to target several different genes, several different probes are preferably used, each preferentially having the ability to hybridize specifically with a different gene.

In the context of the invention, the term “hybridization” is understood to mean the process during which, under appropriate conditions, two nucleotide fragments, such as, for example, a hybridization probe and a target nucleotide fragment, having sequences sufficiently complementary, are capable of forming a double strand with stable and specific hydrogen bonds. A nucleotide fragment which is “able to hybridize” with a polynucleotide is a fragment which can hybridize with said polynucleotide under hybridization conditions which can be determined in each case in a known manner. The hybridization conditions are determined by the stringency, i.e. the strictness of the operating conditions. Hybridization is proportionately more specific the higher the stringency at which it is performed. The stringency is in particular defined on the basis of the base composition of a probe/target duplex, and also by the degree of mismatch between two nucleic acids. The stringency can also be based on the reaction parameters, such as the concentration and type of the ionic species present in the hybridization solution, the nature and the concentration of denaturing agents, and/or the hybridization temperature. The stringency of the conditions under which a hybridization reaction must be performed will mainly depend on the hybridization probes used. All this information is well known and the suitable conditions can be determined by a person skilled in the art. In general, depending on the length of the hybridization probes used, the temperature for the hybridization reaction is between about 20 and 70 ° C, in particular between 35 and 65 ° C in a saline solution at a concentration of about 0.5 to 1 M. A step of detecting the hybridization reaction is then carried out.

In the context of the invention, the expression “enzymatic amplification reaction” designates a process generating multiple copies of a target nucleotide fragment, through the action of at least one enzyme. Such amplification reactions are well known to a person skilled in the art and mention may particularly be made of the following techniques: Polymerase Chain Reaction (PCR), Ligase Chain Reaction (LCR), Repair Chain Reaction (RCR), Self Sustained Sequence Replication (3SR) with application WO-A-90/06995, Nucleic Acid Sequence-Based Amplification (NASBA), Transcription Mediated Amplification (TMA) with U.S. Pat. No. 5,399,491, and Loop Mediated Isothermal Amplification (LAMP) with U.S. Pat. No. 6,410,278. When the enzymatic amplification reaction is a PCR, the term used will more particularly be RT-PCR (RT for “reverse transcription”) when the amplification step is preceded by a step of reverse transcription of messenger RNA (mRNA) to complementary DNA (cDNA), and be qPCR or RT-qPCR when the PCR is quantitative.

Preferably, a subject of the present invention is a method for the in vitro or ex vivo detection of an immunocompromised status in a subject, as defined above and having the following technical characteristics, taken alone or in combination:
step a) comprises quantifying the expression of the TAP2 and IL1R2 target genes;
step a) comprises quantifying the expression of the TAP2 and C3AR1 target genes;
step a) comprises quantifying the expression of the TAP2 and CD177 target genes;
step a) comprises quantifying the expression of the C3AR1 and CD177 target genes;
step a) comprises quantifying the expression of the C3AR1 and IL1R2 target genes;
step a) comprises quantifying the expression of the CD177 and IL1R2 target genes;
step a) comprises quantifying the expression of at least 3 target genes selected from the group consisting of TAP2, C3AR1, CD177 and IL1R2;
step a) comprises quantifying the expression of the TAP2 target gene and of at least one other target gene selected from C3AR1, CD177 and IL1R2;
step a) comprises quantifying the expression of the TAP2 target gene and of at least two other target genes selected from C3AR1, CD177 and IL1R2;
step a) comprises quantifying the expression of the 3 target genes TAP2, C3AR1 and IL1R2;
step a) comprises quantifying the expression of the 3 target genes TAP2, CD3AR1 and CD177;
step a) comprises quantifying the expression of the 3 target genes TAP2, CD177 and IL1R2;
step a) comprises quantifying the expression of the 4 target genes TAP2, C3AR1, CD177 and IL1R2;
the reference value corresponds to the expression of said target gene in a subject with a normal immune status or a subject whose immune status is known to be not immunocompromised;
the immunocompromised status is detected on the basis of at least two variations in expression, said variations being selected from an underexpression of TAP2, an overexpression of C3AR1, an overexpression of CD177, and an overexpression of IL1R2;
the immunocompromised status is detected on the basis of two variations in expression, said variations being selected from an underexpression of TAP2, an overexpression of C3AR1, an overexpression of CD177, and an overexpression of IL1R2;
the immunocompromised status is detected on the basis of three variations in expression, said variations being selected from an underexpression of TAP2, an overexpression of C3AR1, an overexpression of CD177, and an overexpression of IL1R2;
the immunocompromised status is detected on the basis of an underexpression of TAP2, an overexpression of C3AR1, an overexpression of CD177 and an overexpression of IL1R2;
the method of the invention also comprises a step of quantifying the expression of at least one additional target gene selected from the group consisting of CD3D, CIITA, CD74, CTLA4, CX3CR1, IFNγ and S100A9;
the immunocompromised status is then also detected taking into account at least one variation in at least one additional target gene, said variations being selected from an underexpression of CD3D, an underexpression of CIITA, an underexpression of CD74, an underexpression of CTLA4, an underexpression of CX3CR1, an overexpression of IFNγ, and an overexpression of S100A9;
the method of the invention comprises a step of quantifying the expression of all of the following target genes: TAP2, C3AR1, CD177, IL1R2, CD3D, CIITA, CD74, CTLA4, CX3CR1, IFNγ and S100A9;
the subject is a patient, in particular a seriously ill patient in the septic state, more particularly in septic shock;
suffering from burns, more particularly severe burns; suffering from trauma, more particularly severe trauma; or having undergone surgery, more particularly major surgery;
the biological sample is a blood sample;
the biological sample is a whole blood sample;
the biological sample is a sample from a patient in the emergency department, in the resuscitation unit, in an intensive care unit (ICU) or in an ongoing care unit, and preferably from a patient in ICU;
the biological test sample and/or the reference biological sample according to the invention is a blood sample, preferably a whole blood, plasma or serum sample, or a sample of peripheral blood mononuclear cells extracted from a blood sample;
the reference value of a target gene corresponds to the expression of said target gene in a subject with a normal immune status defined by an mHLA-DR value greater than 5000 Ab/C, in particular greater than 5500 Ab/C, advantageously greater than 6000 Ab/C, in particular greater than 6500 Ab/C, preferably greater than 7000 Ab/C, in particular greater than 7500 Ab/C, and most particularly by an mHLA-DR value greater than 8000 Ab/C;
the expression of the target genes is quantified at the RNA or messenger RNA (mRNA) transcript level;
the expression of the target genes is measured at the DNA or complementary DNA (cDNA) level;
the expression of the target genes is measured by the implementation of a method of molecular detection and/or direct quantification by any method known to a person skilled in the art for determining the presence of an mRNA transcript in a sample, such as hybridization methods, preferably with a hybridization chip, by in situ hybridization or by Northern blotting; amplification methods, preferably by Reverse Transcriptase Polymerase Chain reaction (RT-PCR), more preferably by quantitative RT-PCR (RT-qPCR), in particular nested PCR, the PCR reactions can also be multiplexed; sequencing methods, preferably by high throughput sequencing;
the quantification of target gene expression is carried out by RT-PCR, sequencing or hybridization. The quantification is preferably carried out by RT-PCR, and in particular by RT-qPCR;
the expression of the target genes is measured by implementation of a method of indirect detection and/or quantification of the mRNA transcript after conversion of the latter into DNA, or after amplification of said transcript or after amplification of the DNA obtained after conversion of said mRNA transcript into DNA;
the expression of the target genes is normalized with respect to the expression of one or more housekeeping genes, advantageously with respect to the expression of one of the housekeeping genes selected from the group consisting of DECR1, HPRT1, PPIB, GAPDH and ACTB, and combinations thereof;
the expression of the target genes is normalized with respect to the combination of the expression of the housekeeping genes DECR1, HPRT1 and PPIB;
the method according to the invention makes it possible to identify the immunocompromised status in a subject with a sensitivity of at least 70%, and a specificity of at least 80%;
the method of the invention further comprises a step of administering a treatment, preferably an immunomodulatory treatment and/or an anti-inflammatory treatment, suited to the individual's immune status;
the immunostimulant treatment is selected from the group consisting of interleukins, in particular IL-7, IL-15 or IL-3; growth factors, in particular GM-CSF; interferons, in particular IFNγ; Toll agonists; antibodies, particularly anti-PD1, anti-PDL1, anti-LAG3, anti-TIM3, anti-IL-10 or anti-CTLA4 antibodies; transferrins and apoptosis-inhibiting molecules; FLT3L; Thymosin α1; adrenergic antagonists;
the anti-inflammatory treatment is selected from the group consisting of glucocorticoids, cytostatic agents, molecules acting on immunophilins and cytokines, molecules which block the IL-1 receptor and anti-TNF treatments.

According to the invention:
the CD3D gene (CD3 Delta Subunit of T-Cell Receptor Complex) codes for a protein constitutive of the T lymphocyte receptor/CD3 complex (TCR/CD3 complex) and is involved in the development of T lymphocytes and signal transduction. The protein coded for by this gene is the delta subunit of the CD3 complex and, along with four other CD3 subunits, binds either to the TCR alpha/beta or to the TCR gamma/delta to form the TCR/CD3 complex the surface of T lymphocytes. The chromosomal location of CD3D is as follows: chr11: 118,338,954-118,342,744;
the CIITA gene (Class II Major Histocompatibility Complex Transactivator) codes for a transactivator of the class II major histocompatibility complex. The protein, once in the cell nucleus, would act as a co-upregulator for the transcription of genes of the class II major histocompatibility complex, a major component of immune defense. The chromosomal location of CIITA is as follows: chr16: 10,866,212-10,943,021;
the CD74 gene (Invariant Polypeptide of Major Histocompatibility Complex, Class II Antigen-Associated or HLA class II histocompatibility antigen gamma chain) codes for a protein that associates with the class II major histocompatibility complex (MHC) and is an important chaperone protein that regulates antigen presentation for the immune response. It also serves as a cell surface receptor for macrophage cytokine migration inhibitory factor (MIF) which, when bound to the protein coded for by the CD74 gene, initiates cell survival pathways and cell proliferation. This protein also interacts with amyloid precursor protein (APP) and suppresses the production of amyloid beta or β-amyloid peptide. The chromosomal location of CD74 is as follows: chr5: 150,400,041-150,412,936;
the CTLA4 (or CTLA-4; Cytotoxic T-Lymphocyte Associated Protein 4) gene is part of the immunoglobulin superfamily and codes for a protein which transmits an inhibitory signal to T lymphocytes. The chromosomal location of CTLA4 is as follows: chr2: 203,867,771-203,873,965;
the CX3CR1 (C-X3-C motif chemokine receptor 1) gene codes for the fractalkine receptor (or CX3CL1). Fractalkine is a transmembrane protein and a chemokine particularly involved in leukocyte adhesion and migration. The chromosomal location of CX3CR1 is as follows: chr12: 8,058,302-8,066,471;
the IFNγ (interferon γ) gene codes for a soluble cytokine belonging to the type II interferon class. The protein coded for is secreted by cells of the innate and adaptive immune systems. The active protein is a homodimer which binds to the interferon gamma receptor that triggers a cellular response to viral and microbial infections. The chromosomal location of IFNG is as follows: chr12: 68, 154,768-68, 159,741; and
the S100A9 (S100 Calcium Binding Protein A9 or MRP14 for migration inhibitory factor-related protein 14) gene codes for a protein belonging to the S100 protein family containing 2 calcium binding motifs in the EF hand (i.e. a structural domain or a helix-loop-helix motif). The chromosomal location of S100A9 is as follows: chr12: 68, 154,768-68, 159,741.

Another subject of the present invention relates to the use for the in vitro or ex vivo detection of an immunocompromised status in a subject, of quantification, in a biological sample from said subject, expression of at least 2 target genes selected from the group consisting of TAP2, C3AR1, CD177 and IL1R2, and comparison of the expression of each of the genes respectively to a reference value so as to identify the presence or absence of variation in expression, as described above. The use according to the invention further comprises: a step of quantifying the expression of at least one other target gene selected from the group consisting of CD3D, CIITA, CD74, CTLA4, CX3CR1, IFNγ and S100A9.

Another subject of the present invention also relates to a kit for the implementation of the method for the in vitro or ex vivo detection of an immunocompromised status in a subject, as defined above, comprising means for amplifying and/or means for detecting the expression of at least 2 target genes selected from the group consisting of TAP2, C3AR1, CD177 AND IL1R2.

Preferably, a subject of the present invention is a kit for implementing the method for the in vitro or ex vivo detection of an immunocompromised status in a subject, as defined having the following technical features, taken alone or in combination:
said kit comprises the means for amplifying and/or means for detecting the expression of least 3 target genes selected from the group consisting of TAP2, C3AR1, CD177 and IL1R2;
the kit comprises the means for amplifying and/or means for detecting the expression of the 4 target genes TAP2, C3AR1, CD177 and IL1R2;
the kit further comprises means for amplifying and/or means for detecting the expression of at least one other target gene selected from the group consisting of CD3D, CIITA, CD74, CTLA4, CX3CR1, IFNγ and S100A9, and combinations thereof;
the kit comprises the means for amplifying and/or means for detecting the expression of the following 11 target genes: TAP2, C3AR1, CD177, IL1R2, CD3D, CIITA, CD74, CTLA4, CX3CR1, IFNγ and S100A9;
the amplification and/or detection means are reagents specific for the expression products of said target genes, selected from amplification primers or hybridization probes;
the kit further comprises a biological test sample (positive control sample) calibrated for containing the amounts of mRNAs of the TAP2, C3AR1, CD177 and IL1R2 target genes which correspond to the amount or concentration representative of the expression level measured in a sample pool from a subject with an immunocompetent immune status with an mHLA-DR value greater than 5000 Ab/C, in particular greater than 5500 Ab/C, advantageously greater than 6000 Ab/C, in particular greater than 6500 Ab/C, preferably greater than 7000 Ab/C, in particular greater than 7500 Ab/C, and most particularly with an mHLA-DR value greater than 8000 Ab/C.

The present invention also relates to the use of a kit as described above, for the in vitro or ex vivo detection of an immunocompromised status in a subject, preferably a patient, in particular a seriously ill patient in a septic state (more particularly, in septic shock), suffering from burns (more particularly severe burns), trauma (more particularly severe trauma), or having undergone surgery (more particularly major surgery).

### FIGURES

FIG. 1: Distribution of expression levels of various markers in groups of samples defined according to the immune status of the patients. Normalized expression values of all the genes were significantly different between the immune status groups (Mann-Whitney test, p<0.001). n=1183 samples in the group with mHLA-DR>8000 Ab/C (immunocompetent) and n=658 samples in the group with mHLA-DR<8000 Ab/C (immunocompromised).

FIG. 2: Concordance between mHLA-DR categories and stratification by the target genes of the pouch prototype on all subjects (patients and healthy; n=1300). The sample groups based on the mHLA-DR value (<8000 Ab/C for the immunocompromised state and/or 8000 Ab/C for the immunocompetent state) are shown on the left and the stratification using the set of target genes is shown on the right.

FIG. 3: Concordance between mHLA-DR categories and stratification by the target genes of the pouch prototype on all patients (n=541). The sample groups based on the mHLA-DR value (<8000 Ab/C for the immunocompromised state and/or 8000 Ab/C for the immunocompetent state) are shown on the left and the prediction using the set of target genes is shown on the right.

### IMPLEMENTATION EXAMPLES

### Example 1: Ex Vivo Detection of an Immunocompromised Status in a Subject by Implementing the Method of the Invention

### 1. Materials and Methods

1.1. Patients and samples

The patients and samples come from the REALISM study, in which 377 seriously ill patients of various etiologies (sepsis, trauma, elective surgery, burns) were recruited, and also 175 healthy volunteers. Whole peripheral blood was collected in EDTA tubes and in PAXgene blood RNA tubes at different times between study enrollment and day 60 (D60), with up to 7 samples per patient. Samples were taken once from healthy volunteers.

1.2. Measuring mHLA-DR

The number of HLA-DR molecules per monocyte was determined using the BD Quantibrite standardized method (HLA-DR: 340827; Quantibrite: 340495; Becton Dickenson, New Jersey, USA) on fresh EDTA blood samples, within 3 hours of collection, as described above.

### 1.3. Quantification of Target Gene Expression

The PAXgene samples were stabilized for at least 2 hours after collection at ambient temperature and frozen at −80 ° C according to the manufacturer's recommendations. The RNA was then isolated using the Maxwell® HT simplyRNA kit (AX2420, Promega Corporation, Madison, WI, USA). The RNA concentration was determined using the QuantiFluor RNA system (E3310, Promega) on the GloMax® Discover microplate reader (Promega). The integrity of the RNA was evaluated using the RNA 6000 Nano kit (Agilent Technologies, Santa Clara, Calif., USA) on the Agilent 2100 bioanalyzer (Agilent Technologies).

The samples were tested using a FilmArray® pouch prototype optimized to detect, by nested PCR, the TAP2, C3AR1, CD177 and IL1R2 target genes and the additional target genes CD3D, CIITA, CD74, CTLA4, CX3CR1, IFNγ and S100A9.

The expression level of the mRNAs of the target genes was determined on the pouch prototype on the FilmArray® Torch instrument (BioFire®, USA), following the manufacturer's recommendations, by injecting 200 ng of RNA into said pouch prototype. The expression level results are obtained in an automated manner, in less than an hour, before being compiled for analysis. The normalized expression values for the target markers (relative to the DECR1, HPRT1 and PPIB reference genes) were calculated and used for the analyses.

### 1.4. Statistical Analysis

Model for sample identification with mHLA-DR<8000 Ab/C: All samples for which mHLA-DR measurements were available were used in the analysis. The samples were considered immunocompromised when the quantification of mHLA-DR is less than 8000 Ab/C, otherwise these samples were considered immunocompetent. The data was divided into a training set containing 1300 samples (70%) and a test set containing 541 samples (30%). The distribution between the training set and the test set was balanced according to the distribution of the mHLA-DR values, from the time of sampling, and to the patient etiology. A PLS (Partial Least Squares) regression model combining the expression level of the markers of the pouch prototype was trained by repeated cross-validation 20X-5 times for the prediction of mHLA-DR less than 8000 Ab/C. SMOTE (Synthetic Minority Over-Sampling Technique) over-sampling was used to balance the training set between categories. The number of components was evaluated manually from 1 up to the maximum possible. The model performance was evaluated on the test set by calculating the area under the ROC curve (AUC), the accuracy, the sensitivity, the specificity, the positive predictive value and the negative predictive value.

The statistical analyses were carried out using the R software, version 3.6.2.

### 2. Results

The mHLA-DR data and target gene expression data were both available for 1841 samples distributed as follows: 163 samples from healthy volunteers and 1678 samples from patients corresponding to 106 sepsis, 136 trauma, 109 surgeries and 24 burns.

Overall, the mHLA-DR values ranged from 439 to 80 066 Ab/C, with 36% of the values less than 8000 Ab/C.

The detection of variations in the expression of the pouch prototype markers (target and additional genes) according to the invention was carried out.

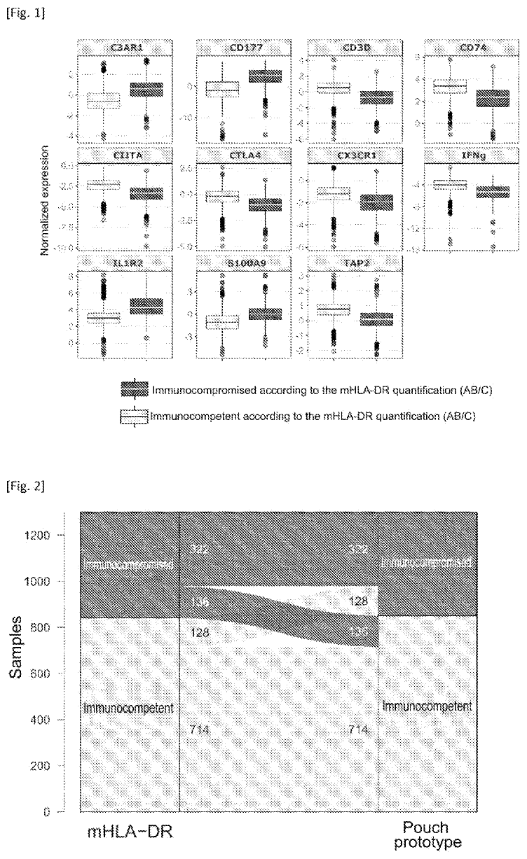

The data are shown in FIG. 1. In the immunocompromised group, down-regulation of the expression of: CD3D, CD74, CIITA, CTLA4, CX3CR1, IFNγ and TAP2 was observed; while the following markers had their expression up-regulated: C3AR1, CD177, IL1R2, S100A9.

The individual normalized expression levels of the TAP2, C3AR1, CD177 and IL1R2 mRNA markers, and also those of the different combinations of the markers TAP2, C3AR1, CD177, IL1R2, CD3D, CIITA, CD74, CTLA4, CX3AR1, IFNγ and S100A9 were combined in a trained model for the identification of immunocompromised patients (mHLA-DR<8000 Ab/C).

Performance data for the different combinations of target genes according to the invention are shown in table 2.

TABLE 2

| | | | | Negative | Positive |
|---|---|---|---|---|---|
| | | | | predictive | predictive |
| | AUC | | | value | value |
| Markers | [CI 95%] | Sensitivity | Specificity | (NPV) | (PPV) |
| TAP2; C3AR1 | 0.85 | 0.71 | 0.83 | 0.83 | 0.71 |
| | (0.82-0.88) | | | | |
| TAP2; IL1R2 | 0.85 | 0.68 | 0.85 | 0.82 | 0.73 |
| | (0.81-0.88) | | | | |
| TAP2; CD177 | 0.82 | 0.70 | 0.82 | 0.82 | 0.69 |
| | (0.78-0.85) | | | | |
| C3AR1; CD177 | 0.82 | 0.72 | 0.80 | 0.83 | 0.68 |
| | (0.79-0.86) | | | | |
| C3AR1; IL1R2 | 0.82 | 0.65 | 0.82 | 0.80 | 0.68 |
| | (0.79-0.86) | | | | |
| CD177; IL1R2 | 0.82 | 0.71 | 0.79 | 0.82 | 0.66 |
| | (0.78-0.85) | | | | |
| C3AR1; | 0.86 | 0.73 | 0.83 | 0.84 | 0.71 |
| IL1R2; TAP2 | (0.83-0.89) | | | | |
| C3AR1; | 0.83 | 0.73 | 0.81 | 0.84 | 0.69 |
| IL1R2; CD177 | (0.80-0.87) | | | | |
| IL1R; CD177; | 0.84 | 0.71 | 10.81 | 0.82 | 0.69 |
| TAP2 | (0.81-0.88) | | | | |
| C3AR1; CD177; | 0.85 | 0.71 | 0.81 | 0.83 | 0.68 |
| TAP2 | (0.81-0.88) | | | | |
| C3AR1; IL1R2; | 0.86 | 0.74 | 0.81 | 0.84 | 0.70 |
| CD177; TAP2 | (0.83-0.89) | | | | |
| Pouch prototype | 0.87 | 70% | 85% | 84% | 72% |
| (TAP2; C3AR1; | (0.85-0.89) | | | | |
| CD177; IL1R2; | | | | | |
| CD3D; CIITA; CD74 | | | | | |
| CTLA4; CX3AR1; | | | | | |
| IFNγ; | | | | | |
| S100A9) on training | | | | | |
| set | | | | | |
| (n = 1300) | | | | | |
| Pouch prototype | 0.86 | 72% | 85% | 84% | 73% |
| (TAP2; C3AR1; | (0.82-0.89) | | | | |
| CD177; IL1R2; | | | | | |
| CD3D; CIITA; CD74 | | | | | |
| CTLA4; CX3AR1; | | | | | |
| IFNγ; | | | | | |
| S100A9) on | | | | | |
| test set (n = 541) | | | | | |

The model obtained very good performances for the identification of patients with an immunocompromised status, with an AUC greater than 0.82 for all the gene sets tested.

By way of comparison, the performance obtained on the test set with CD74 (which represents the γ-invariant chain of HLA-DR) as the sole substitute for mHLA-DR in a generalized linear model was lower, with an AUC of 0.77 (CI 95% [0.73-0.81]), thus demonstrating the great advantage of the combinations of target genes according to the invention.

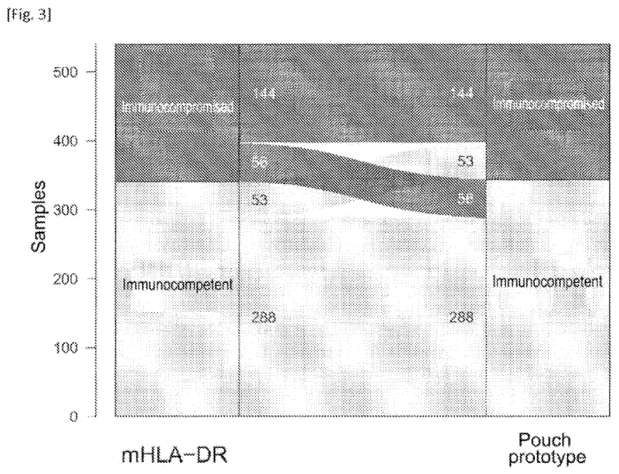

Agreement was performed between the mHLA-DR results and the identification with the target genes on the training set (n=1300 samples; FIG. 2) and on the test set (n=541; FIG. 3). The sample groups based on the mHLA-DR value (<8000 Ab/C for immunocompromised and/or 8000 Ab/C for immunocompetent) are shown on the left and the prediction using the set of target genes according to the invention is shown on the right. It appears that the agreement obtained in all the samples between the mHLA-DR model and the target genes is 80%, whether on the training set or the test set.

### 3. Conclusion

It is clearly apparent that the method of the invention for the in vitro or ex vivo detection of an immunocompromised state in a subject is effective and appears to be an excellent alternative to the mHLA-DR quantification, since the correlation of the pouch prototype data and mHLA-DR data shows similarity in the identification of immunocompromised or immunocompetent patients.

Even more importantly, the method of the invention has several other advantages. For example, the pre-analytical process is facilitated by stabilizing whole blood RNA in the PAXgene tube, requiring no sample preparation. In addition, the results are obtained in one hour, without manipulation, by virtue of its implementation on the fully automated platform such as FilmArray® (BioFire®), already widely used in clinics for microbiological diagnosis. At the very least, the method according to the invention thus allows early identification of the immunocompromised status of a patient, in the time for example that an mHLA-DR measurement can be performed.

Despite good agreement between the data from the mHLA-DR quantification and those from the method of the invention, some samples are misclassified. This could be due to the intrinsic variability of the two techniques. Another explanation could be the differences in regulation between a protein (mHLA-DR) and the expression of several mRNA markers. In addition, while HLA-DR is measured at the cellular level only in monocytes, the method of the invention allows quantification at the mRNA level in whole blood, comprising monocytes but also other cells expressing HLA-DR (for example B lymphocytes), and thus captures other immune modifications, not necessarily related to the regulation of mHLA-DR. However, this does not in any way detract from the advantages of the method according to the invention for the identification of an immunocompromised status in a subject.

1. A method for the in vitro or ex vivo detection of an immunocompromised status in a subject, comprising the following steps:
a. quantifying, in a biological sample from said subject, the expression of at least 2 target genes selected from the group consisting of TAP2, C3AR1, CD177 and IL1R2;
b. comparing the expression of each of the genes respectively with a reference value so as to identify the presence or absence of a variation in the expression, the reference value corresponding to the expression of said target gene in a subject with a normal immune status defined by an mHLA-DR greater than 5000 cell-bound antibodies (Ab/C), the immunocompromised status being detected on the basis of at least two variations in the expression, said variations being selected from:
an underexpression of TAP2,
an overexpression of C3AR1,
an overexpression of CD177, and
an overexpression of IL1R2.

2. The method as claimed in claim 1, wherein step a) comprises quantifying the expression of at least 3 target genes selected from the group consisting of TAP2, C3AR1, CD177 and IL1R2.

3. The method as claimed in claim 1, wherein step a) comprises quantifying the expression of the TAP2 target gene and of at least one other target gene selected from C3AR1, CD177 and IL1R2.

4. The method as claimed in claim 1, wherein step a) comprises quantifying the expression of the 4 target genes TAP2, C3AR1, CD177 and IL1R2.

5. The method as claimed in claim 1, wherein it further comprises a step of quantifying the expression of at least one other target gene selected from the group consisting of CD3D, CIITA, CD74, CTLA4, CX3CR1, IFNγ and S100A9, wherein the immunocompromised status is detected taking into account at least one additional variation in the expression, said additional variation being selected from:
an underexpression of CD3D,
an underexpression of CIITA,
an underexpression of CD74,
an underexpression of CTLA4,
an underexpression of CX3CR1,
an overexpression of IFNγ, and
an overexpression of S100A9.

6. The method as claimed in claim 1, wherein the biological sample is a blood sample.

7. The method as claimed in claim 1, wherein the biological sample is from a patient in the emergency department, in the resuscitation unit, in an intensive care unit (ICU) or in an ongoing care unit.

8. The method as claimed in claim 1, wherein the expression is measured at the RNA or messenger RNA level.

9. The method as claimed in claim 1, wherein the expression is measured by RT-PCR, by sequencing or by hybridization.

10. The method as claimed in claims, claim 1, wherein the expression is normalized with respect to the expression of one or more housekeeping genes selected from the group consisting of DECR1, HPRT1, PPIB, GAPDH and ACTB.

11. A kit for the implementation of the method as claimed in claim 1, comprising means for amplifying and/or means for detecting the expression of at least 2 target genes selected from the group consisting of TAP2, C3AR1, CD177 and IL1R2.

12. The kit as claimed in claim 11, wherein it comprises means for amplifying and/or means for detecting the expression of at least 3 target genes selected from the group consisting of TAP2, C3AR1, CD177 and IL1R2.

13. The kit as claimed in claim 11, further comprising means for amplifying and/or means for detecting the expression of at least one other target gene selected from the group consisting of CD3D, CIITA, CD74, CTLA4, CX3CR1, IFNγ and S100A9.

14. The kit as claimed in claim 1, wherein the amplification and/or detection means are reagents Specific for the expression products of said target genes, selected from amplification primers or hybridization probes.
